(19) United States
(12) Patent Application Publication
(10) Pub. No.: US 2006/0211726 A1
(43) Pub. Date: Sep. 21, 2006

(54) Ascomycin derivatives for combination treatment with tazorotene

(76) Inventor: Frederick Beddingfield, Pacific Palisades, CA (US)

Correspondence Address: BRENT A. JOHNSON;ALLERGAN, INC., 2525 Dupont Drive, T2-7H, Irvine, CA, 92612, US

(21) Appl. No.: 11/385,600

(22) Filed: Mar. 21, 2006

Related U.S. Application Data
(60) Provisional application No. 60/664,077, filed on Mar. 21, 2005.

(51) Int. Cl. A61K 31/4745; A61K 31/4436

(52) U.S. Cl. 514/291; 514/337

(57) ABSTRACT

A method is disclosed herein comprising administering a therapeutically effective amount of an ascomycin derivative topically in combination with tazarotene, wherein said method is effective in reducing adverse events associated with tazarotene. A method is also disclosed herein comprising administering a therapeutically effective amount of an ascomycin derivative topically in combination with tazarotene to a person for the treatment or prevention of psoriasis. Thus, a composition comprising a therapeutically effective amount of tazarotene and an ascomycin derivative is disclosed herein.

### CROSS REFERENCE TO RELATED APPLICATIONS

This is a non-provisional application which claims the benefit of Provisional Application No. 60/664,077 filed Mar. 21, 2005 and incorporated in its entirety herein.

### BACKGROUND OF THE INVENTION

### DESCRIPTION OF RELATED ART

Proliferative skin diseases are widespread throughout the world and afflict millions of humans and their domesticated animals. This disclosure provides a method for treatment of such diseases. As used hereinafter in this specification and in the claims, the expression “proliferative skin diseases” means benign and malignant proliferative skin diseases which are characterized by accelerated cell division in the epidermis, dermis or appendages thereto, associated with incomplete tissue differentiation. Such diseases include: psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant sun-induced keratosis, non-malignant keratosis, acne, and seborrhic dermatitis in humans and atopic dermatitis in domesticated animals.

Heretofore, proliferative skin diseases have been generally accepted by mankind as an ongoing evil having degrees of severity variable with inherited skin traits and external factors but always have been recognized as unsightly, painful, morbid diseases. Over the history of mankind innumerable medicines and treatments have been proposed, tried and used with varying degrees of success.

Tazarotene is sold by Allergan, Inc. under the tradename Tazorac® for the treatment of psoriasis.

Klaus teaches the use of pimecrolimus for the treatment of psoriasis (Dermatologic Clinics (2004), 22(4), 461-465). Scheinfeld discusses the utility of tacrolimus and pimecrolimus in treating psoriasis (Dermatology Online Journal, 10(1):3.) Pimecrolimus is marketed by Novartis under the tradename Elidel® for the treatment of eczema.

Tacrolimus is marketed by Fujisawa as tacrolimus under the tradename Protopic® for the treatment of eczema.

### DETAILED DESCRIPTION OF THE INVENTION

A method is disclosed herein comprising administering a therapeutically effective amount of an ascomycin derivative topically in combination with tazarotene, wherein said method is effective in reducing adverse events associated with tazarotene. Said adverse events may include mild to moderate local irritation including pruritus, erythema and burning skin.

A method is also disclosed herein comprising administering a therapeutically effective amount of an ascomycin derivative topically in combination with tazarotene to a person for the treatment or prevention of psoriasis.

Ascomycin is the compound depicted below.

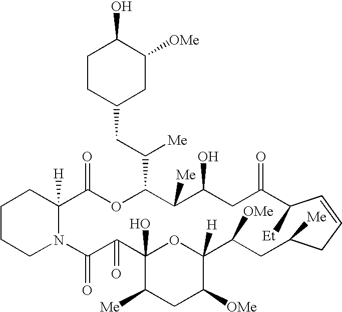

An “ascomycin derivative” is a derivative of ascomycin, ascomycin itself, or a prodrug or metabolite of ascomycin or a derivative thereof. In one embodiment, there is a difference of less than 5 heavy atoms between the derivative and ascomycin, and all the cycles are intact. A heavy atom is an atom which is not hydrogen. In other words, less than 5 heavy atoms are added to the structure to give the derivative. Alternatively, less than 5 heavy atoms are removed from the structure. Alternatively, less than 5 heavy atoms are substituted with a different heavy atom.

In another embodiment, the derivative has a difference of less than 2 heavy atoms from ascomycin.

In another embodiment, the ascomycin derivative is selected from the group consisting of tacrolimus and pimecrolimus.

In one method the ascomycin derivative is pimecrolimus.

In another method the ascomycin derivative is tacrolimus.

Administration of tazarotene and the ascomycin derivative may be carried out by administering two separate compositions, one composition containing each drug. Alternatively, a composition comprising both tazarotene and the ascomycin derivative may be administered.

Thus, a composition comprising a therapeutically effective amount of tazarotene and an ascomycin derivative is disclosed herein.

A therapeutically effective amount of tazarotene and the ascomycin derivative can be easily determined by a person of ordinary skill in the art. One composition comprises from 0.01% to 0.2% tazarotene.

Another composition comprises from 0.05% to 0.15% tazarotene.

Another composition comprises about 0.1% tazarotene.

Another composition comprises about 0.05% tazarotene.

Another composition comprises pimecrolimus.

Another composition comprises about 1% pimecrolimus.

One composition comprises from 0.1% to 2% pimecrolimus.

Another composition comprises from 0.5% to 1.5% pimecrolimus.

Another composition comprises about 1% pimecrolimus and about 0.1% tazarotene.

Another composition comprises about 1% pimecrolimus and about 0.05% tazarotene.

Another composition comprises tacrolimus.

Another composition comprises about 0.1% tacrolimus.

Another composition comprises from 0.005% to 0.2% tacrolimus.

Another composition comprises from 0.01% to 0.15% tacrolimus.

Another composition comprises about 0.1% tacrolimus and about 0.1% tazarotene.

Another composition comprises about 0.1% tacrolimus and about 0.05% tazarotene.

Another composition comprises about 0.03% tacrolimus.

Another composition comprises about 0.03% tacrolimus and about 0.1% tazarotene.

Another composition comprises about 0.03% tacrolimus and about 0.05% tazarotene.

Another embodiment is use of a therapeutically effective amount of tazarotene and ascomycin derivative in the manufacture of a medicament for the treatment of psoriasis.

In another embodiment, the medicament is for topical administration.

One medicament comprises from 0.01% to 0.2% tazarotene.

Another medicament comprises from 0.05% to 0.15% tazarotene.

Another medicament comprises about 0.1% tazarotene.

Another medicament comprises about 0.05% tazarotene.

Another medicament comprises pimecrolimus.

Another medicament comprises about 1% pimecrolimus.

One medicament comprises from 0.1% to 2% pimecrolimus.

Another medicament comprises from 0.5% to 1.5% pimecrolimus.

Another medicament comprises about 1% pimecrolimus and about 0.1% tazarotene.

Another medicament comprises about 1% pimecrolimus and about 0.05% tazarotene.

Another medicament comprises tacrolimus.

Another medicament comprises about 0.1% tacrolimus.

Another medicament comprises from 0.005% to 0.2% tacrolimus.

Another medicament comprises from 0.01% to 0.15% tacrolimus.

Another medicament comprises about 0.1% tacrolimus and about 0.1% tazarotene.

Another medicament comprises about 0.1% tacrolimus and about 0.05% tazarotene.

Another medicament comprises about 0.03% tacrolimus.

Another medicament comprises about 0.03% tacrolimus and about 0.1% tazarotene.

Another medicament comprises about 0.03% tacrolimus and about 0.05% tazarotene.

Another embodiment is use of tazarotene with another compound in the manufacture of a medicament for the treatment of psoriasis, wherein said compound is an ascomycin derivative.

In another such use, said compound is pimecrolimus.

In another such use, the concentration of pimecrolimus is about 1%.

In another such use, said compound is tacrolimus.

In another such use, the concentration of tacrolimus is about 0.1% or about 0.03%.

In another such use, the concentration of tazarotene is about 0.05% or about 0.1%.

Another embodiment is a composition comprising 0.1% or 0.05% tazarotene; and 1% pimecrolimus, 0.03% tacrolimus, or 0.1% tacrolimus.

For the purposes of this specification and the claims, a proliferative skin disease is alleviated when there is a noticeable decrease in the thickness of a lesion to palpation, with or without residual redness, or residual slightly dilated blood vessels or residual hyper- or hypo-pigmentation. For purposes of this disclosure and the claims, psoriasis is alleviated when a scale-free psoriasis lesion is noticeably decreased in thickness, or noticeably but incompletely cleared or completely cleared.

The compositions utilized disclosed herein may be applied topically.

The term “topical” as employed herein relates to the use of the active ingredient incorporated in a suitable pharmaceutical carrier, and applied at the site of the disease for exertion of local action. Accordingly, such topical compositions include those pharmaceutical forms in which the compound is applied externally by direct contact with the skin surface to be treated. Conventional pharmaceutical forms for this purpose include ointments, lotions, pastes, jellies, sprays, aerosols, bath oils and the like. The term “ointment” embraces formulations (including creams) having oleaginous, absorption, water-soluble and emulsion-type bases, e.g., petroleum, lanolin, polyethylene glycols, as well as mixtures thereof. Topical application with occlusion of an area larger than the medicated area may produce improved results relative to non-occluded topical applications.

Tazarotene may utilized as a stable gel formulation for topical treatment of skin conditions in humans, said stable gel formulation comprising: Ethyl-6-[2-(4,4-dimethylthiochroman-6-yl)ethynyl]nicotinate in a plurality of nonaqueous vehicles for both solubilizing tazarotene and forming a gel therewith, said nonaqueous vehicles enabling topical application of the gel to a skin condition, said plurality of vehicles each being present in amounts, and in combination, to control release of tazarotene from the gel to the skin conditions. In the tazarotene formulation the vehicles are present in amounts selected to produce maximum release of the active agent, i.e. tazarotene, from the gel when all the vehicles are present therein. Preferably the formulation comprises three vehicles and more preferably the formulation comprises three vehicles which are used to both solubilize the active agent and form a gel.

The formulation preferably comprises the three vehicles, e.g. Polysorbate 40, Poloxamer 407 and Hexylene glycol. Polysorbate 40 is

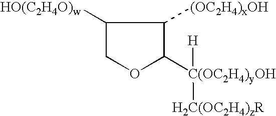

wherein the Sum of w, x, y, and z is 20 and R is (C₁₅H₃₁)COO and Poloxamer 407 is HO(C₂H₄O)ₐ(C₃H₆O)_{b}(C₂H₄O)ₐH having the following properties.

USAN for Poloxamers

| | | | | | BASF |
|---|---|---|---|---|---|
| | | | | | Corp. |
| | | Average | Average | | Brand |
| | Physical | Molecular | Values | | Name |
| Poloxamer | Form | Weight | a | b | Pluronic |
| 124 | Liquid | 2090 to 2360 | 12 | 20 | L 44 |
| 188 | Solid | 7680 to 9510 | 80 | 27 | F 68 |
| 237 | Solid | 6840 to 8830 | 64 | 37 | F 87 |
| 338 | Solid | 12700 to 17400 | 141 | 44 | F 108 |
| 407 | Solid | 9840 to 14600 | 101 | 56 | F 127 |

More preferably, tazarotene is utilized as a stable gel formulation for topical treatment of psoriasis comprising an effective amount of Ethyl-6-[2-(4,4-dimethylthiochroman-6-yl)ethynyl]nicotinate in a pharmaceutical carrier comprising:
(a) water;
(b) edetate disodium;
(c) ascorbic acid;
(d) Carbomer 934P;
(e) Poloxamer 407;
(f) polyethylene glycol;
(g) Polysorbate 40;
(h) hexylene glycol;
(i) butylated hydroxytoluene;
(j) butylated hydroxyanisole;
(k) benzyl alcohol; and
(l) tromethamine.

The tazarotene formulation may comprise Polysorbate 40 in an amount up to about 0.4% by weight, Poloxamer 407 in an amount up to about 0.4% by weight, and hexylene glycol in an amount up to about 2% by weight or more preferably Polysorbate 40, in an amount of about 0.32% by weight, Poloxamer 407 in an amount of about 0.18% by weight, and hexylene glycol in an amount of about 2% by weight.

Most preferably, the tazarotene formulation comprises:

| | | CONCENTRATION |
|---|---|---|
| INGREDIENT | FUNCTION | % W/W |
| tazarotene | Drug | 0.1 |
| purified water | Excipient | 49.25 |
| Edetate Disodium | Stabilizer | 0.05 |
| Ascorbic acid | Stabilizer | 0.05 |
| Carbomer 934P¹ | Thickening | 1.25 |
| | agent | |
| Poloxamer 407 | Surfactant | 0.2 |
| PEG-400 | Co-solvent | 45.0 |
| Polysorbate 40 | Surfactant | 0.2 |
| Hexylene glycol | Co-solvent | 2.0 |
| Butylated | Stabilizer | 0.05 |
| hydroxytoluene | | |
| Butylated | Stabilizer | 0.05 |
| hydroxyanisole | | |
| Benzyl alcohol | Preservative | 1.0 |
| Triethanolamine/ | Neutralizer | 0.8 |
| Tromethamine | | |

¹Carbomer 934P [1975]. NF. The viscosity of a neutralized 0.5 percent aqueous dispersion of Carbomer 934P is between 29,400 and 39,400 centiposes. (1) Polymer of 2-propenoic acid, cross-linked with allyl ethers of sucrose or pentaerythritol; (2) Polymer of acrylic acid, cross-linked with allyl ethers of sucrose or pentaerythritol. Molecular weight is approximately 3,000,000.

The tazarotene formulation and the ascomycin derivative formulation, each, will be applied, topically, in an amount to achieve the maximum effect on alleviating the proliferative skin disease symptoms without causing an adverse reaction. Selection of such an amount of either formulation is well within the skill of the art.

Preferably, the tazarotene formulation is utilized to provide from about 0.5 to about 5 mg of tazarotene per cm² of affected skin, more preferably from about 1 to about 3 mg/cm², e.g. 2 mg/cm².

Pimecrolimus is commercially available in a 1% cream from Novartis under the tradename Elidel®.

Tacrolimus is commercially available from Fujisawa as a 0.1% or a 0.03% ointment under the tradename Protopic®.

The invention is further illustrated by the following examples which are illustrative of various aspects of the invention, and are not intended as limiting the scope of the invention as defined by the appended claims.

### EXAMPLE 1

A composition comprising both tazarotene and pimecrolimus may be prepared according to the formula below.

| | | CONCENTRATION |
|---|---|---|
| INGREDIENT | FUNCTION | % W/W |
| tazarotene | Drug | 0.1 |
| pimecrolimus | Drug | 1% |
| purified water | Excipient | 49.25 |
| Edetate Disodium | Stabilizer | 0.05 |
| Ascorbic acid | Stabilizer | 0.05 |
| Carbomer 934P¹ | Thickening | 1.25 |
| | agent | |
| Poloxamer 407 | Surfactant | 0.2 |
| PEG-400 | Co-solvent | 45.0 |
| Polysorbate 40 | Surfactant | 0.2 |
| Hexylene glycol | Co-solvent | 2.0 |
| Butylated | Stabilizer | 0.05 |
| hydroxytoluene | | |
| Butylated | Stabilizer | 0.05 |
| hydroxyanisole | | |
| Benzyl alcohol | Preservative | 1.0 |
| Triethanolamine/ | Neutralizer | 0.8 |
| Tromethamine | | |

¹Carbomer 934P [1975]. NF. The viscosity of a neutralized 0.5 percent aqueousdispersion of Carbomer 934P is between 29,400 and 39,400 centiposes. (1) Polymer of 2-propenoic acid, cross-linked with allyl ethers of sucrose or pentaerythritol; (2) Polymer of acrylic acid, cross-linked with allyl ethers of sucrose or pentaerythritol. Molecular weight is approximately 3,000,000.

### EXAMPLE 2

A composition comprising both tazarotene and tacrolimus may be prepared according to the formula below.

| | | CONCENTRATION |
|---|---|---|
| INGREDIENT | FUNCTION | % W/W |
| tazarotene | Drug | 0.1 |
| tacrolimus | Drug | 0.1% |
| purified water | Excipient | 49.25 |
| Edetate Disodium | Stabilizer | 0.05 |
| Ascorbic acid | Stabilizer | 0.05 |
| Carbomer 934P¹ | Thickening | 1.25 |
| | agent | |
| Poloxamer 407 | Surfactant | 0.2 |
| PEG-400 | Co-solvent | 45.0 |
| Polysorbate 40 | Surfactant | 0.2 |
| Hexylene glycol | Co-solvent | 2.0 |
| Butylated | Stabilizer | 0.05 |
| hydroxytoluene | | |
| Butylated | Stabilizer | 0.05 |
| hydroxyanisole | | |
| Benzyl alcohol | Preservative | 1.0 |
| Triethanolamine/ | Neutralizer | 0.8 |
| Tromethamine | | |

¹Carbomer 934P [1975]. NF. The viscosity of a neutralized 0.5 percent aqueous dispersion of Carbomer 934P is between 29,400 and 39,400 centiposes. (1) Polymer of 2-propenoic acid, cross-linked with allyl ethers of sucrose or pentaerythritol; (2) Polymer of acrylic acid, cross-linked with allyl ethers of sucrose or pentaerythritol. Molecular weight is approximately 3,000,000.

### EXAMPLE 3

A composition comprising both tazarotene and tacrolimus may be prepared according to the formula below.

| | | CONCENTRATION |
|---|---|---|
| INGREDIENT | FUNCTION | % W/W |
| tazarotene | Drug | 0.1 |
| tacrolimus | Drug | 0.03% |
| purified water | Excipient | 49.25 |
| Edetate Disodium | Stabilizer | 0.05 |
| Ascorbic acid | Stabilizer | 0.05 |
| Carbomer 934P¹ | Thickening | 1.25 |
| | agent | |
| Poloxamer 407 | Surfactant | 0.2 |
| PEG-400 | Co-solvent | 45.0 |
| Polysorbate 40 | Surfactant | 0.2 |
| Hexylene glycol | Co-solvent | 2.0 |
| Butylated | Stabilizer | 0.05 |
| hydroxytoluene | | |
| Butylated | Stabilizer | 0.05 |
| hydroxyanisole | | |
| Benzyl alcohol | Preservative | 1.0 |
| Triethanolamine/ | Neutralizer | 0.8 |
| Tromethamine | | |

¹Carbomer 934P [1975]. NF. The viscosity of a neutralized 0.5 percent aqueous dispersion of Carbomer 934P is between 29,400 and 39,400 centiposes. (1) Polymer of 2-propenoic acid, cross-linked with allyl ethers of sucrose or pentaerythritol; (2) Polymer of acrylic acid, cross-linked with allyl ethers of sucrose or pentaerythritol. Molecular weight is approximately 3,000,000.

### EXAMPLE 4

A composition according to one of examples 1-3 is administered topically once a day to person suffering from psoriasis. Improvement of symptoms is observed with greater efficacy and fewer side effects than by using tazarotene alone once a day or the ascomycin derivative alone.

### EXAMPLE 5

Tacrilomus is administered twice a day as 0.1% Protopic® ointment to a person suffering from psoriasis. The same person receives 0.1% tazarotene as Tazorac® ointment once a day. Improvement of symptoms is observed with greater efficacy and fewer side effects than by using 0.1% tazarotene alone once a day or 0.1% tacrilomus alone twice a day.

What is claimed is:

1. Use of tazarotene with another compound in the manufacture of a medicament for the treatment of psoriasis, wherein said compound is an ascomycin derivative.

2. Use of claim 1 wherein said compound is pimecrolimus.

3. Use of claim 2 wherein the concentration of pimecrolimus is about 1%.

4. Use of claim 1 wherein said compound is tacrolimus.

5. Use of claim 4 wherein the concentration of tacrolimus is about 0.1% or about 0.03%.

6. Use according to any of claims 1 to 5 wherein the concentration of tazarotene is about 0.05% or about 0.1%.

7. A composition comprising 0.1% or 0.05% tazarotene; and 1% pimecrolimus, 0.03% tacrolimus, or 0.1% tacrolimus.
